(19) United States
(12) Patent Application Publication
(10) Pub. No.: US 2022/0162652 A1
(43) Pub. Date: May 26, 2022

(54) METHOD FOR PREPARING AN EXTRACT CONTAINING GALLIC ACID, AQUEOUS CONCENTRATE CONTAINING GALLIC ACID, AND FOODSTUFF AND FOOD SUPPLEMENT CONTAINING THE AQUEOUS CONCENTRATE

(71) Applicant: Red Bull GmbH, Fuschl am See (AT)

(72) Inventors: Josef Nachbagauer, Fuschl am See (AT); Marcel Jenny, Fuschl am See (AT); Christian Rinderer, Fuschl am See (AT); Isabella Winter, Fuschl am See (AT)

(21) Appl. No.: 17/456,157

(22) Filed: Nov. 22, 2021

(30) Foreign Application Priority Data
Nov. 20, 2020 (EP) 20209040.3

(51) Int. Cl. C12P 7/42; C07C 63/06; A23L 33/105

(52) U.S. Cl. CPC C12P 7/42; A23V 2002/00; A23L 33/105; C07C 63/06

(57) ABSTRACT

A method for preparing an extract containing gallic acid includes performing an extraction treatment on plants or plant constituents containing tannins by means of water or an aqueous system in the presence of the enzyme tannase to obtain a product mixture containing gallic acid. Further disclosed is a method for preparing an extract containing gallic acid, comprising performing an extraction treatment on plants or plant constituents containing tannins by means of a mixture containing a polar organic solvent and water to obtain a product mixture containing polyphenols, separating the plants or plant constituents from the product mixture to obtain a liquid extract, removing the polar organic solvent from the liquid extract, and treating the aqueous extract with the enzyme tannase to obtain an aqueous system containing gallic acid. In addition, the disclosure includes an aqueous concentrate containing gallic acid and a foodstuff or food supplement comprising the aqueous concentrate.

### BACKGROUND

### Technical Field

The present disclosure relates to a method for preparing an extract containing gallic acid, in particular in high concentrations. Moreover, the disclosure relates to an aqueous concentrate containing gallic acid. Furthermore, the disclosure relates to a foodstuff and food supplement containing the aqueous concentrate according to the disclosure.

### Description of the Related Art

Gallic acid (3,4,5-trihydroxybenzoic acid) is the acid component of gallotannins, which are hydrolyzable tannins. They are found, for example, in oak bark and in oak apples. Gallic acid is used to produce antioxidants, sunscreens, and dyes. Furthermore, it has been postulated that gallic acid, due to its hypoglycemic properties, helps prevent adverse health effects in obese people that consume foods rich in gallic acid. The consumption of gallic acid is said to be associated with demonstrable DNA protection or reduced oxidation of DNA bases as well as reduced inflammation parameters (see also T. Setayesh et al. “Gallic acid, a common dietary phenolic protects against high fat diet induced DNA damage” in European Journal of Nutrition, vol. 58, pages 2315 to 2326, 2019).

According to EP 1 942 919 B 1, a composition containing a polyphenol extract from grapes, which may comprise 400 to 1,500 ppm of gallic acid, is said to be suitable for treating metabolic syndrome or prehypertension.

According to EP 2 095 718 B 1, a tea beverage based on a tea extract, in which the weight ratio of gallic acid to non-polymeric catechins must not exceed 0.3, is characterized by reduced bitterness and a reduced sour taste.

EP 2 143 344 B1 describes a beverage composition containing 0.5 to 25.0% by weight of non-polymeric catechins, a carbohydrate and a hydroxycarboxylic acid. In these compositions, the gallic acid content has to be less than 0.6% by weight.

Previously, gallic acid has often been obtained by way of aqueous extraction methods. However, these methods frequently produce only low yields of gallic acid and are intensive in terms of materials, equipment, and costs.

### DETAILED DESCRIPTION

Described herein is a method for obtaining gallic acid that does not have the drawbacks of the prior art and, in particular, reliably, and efficiently produces good yields of gallic acid.

Thus, a method for preparing an extract containing gallic acid (also called a first embodiment of the method according to the disclosure), comprises:
a) providing, in particular comminuted and/or, in particular and, dried, plants or plant constituents containing tannins, in particular tannic acid,
b) providing the enzyme tannase (IUB 3.1.1.20),
c) providing at least one polar organic solvent,
d) optionally comminuting or further comminuting, respectively, the plants or plant constituents according to step a), and
e) performing an extraction treatment on the plants or plant constituents obtained, in particular comminuted, according to step a) and/or, in particular or, d), by way of water or an aqueous system in the presence of the enzyme tannase (IUB 3.1.1.20) to obtain a product mixture containing gallic acid.

For the, in particular comminuted and/or dried, plants or plant constituents, the following may be used: leaves, wood, bark, fruits, seeds and/or roots, preferably the fruits, of legumes, e.g., beans and peas; nuts, e.g., walnuts (Juglans regia); grains; trees, e.g., oak (Quercus), preferably the cupules and/or fruits (Valonea), particularly preferably from varieties of oak growing in Asia Minor and/or in the southern Balkans, and/or oak galls (Galla), in particular from Quercus infectoria, Quercus petraea or Quercus robur, spruces (Picea), the carob tree (also referred to as St John's bread) (Ceratonia siliqua), the Divi-Divi-tree (Caesalpinia coriaria), the kaki tree (Diospyros kaki), the sorb tree (Sorbus domestica), acacia (Acacieae), e.g., cutchtree (also referred to as catechu) (Senegalia catechu), willow (Salix), Myrobalan tree, mangrove tree (Rhizophora), mango tree (Mangifera indica), in particular bark, fruit pulp and/or kernel of the mango tree, hemlock (Tsuga), the urunday tree (Astronium balansae), birch (Betula), Elaeocarpus, in particular the outer bark thereof, tree of heaven (Ailanthus altissima), chestnut (Castanea), in particular the fruits and leaves from chestnut, the betel nut palm (Areca catechu), the cashew tree (Anacardium occidentals) and/or quebracho (Aspidosperma quebracho-blanco); mimosa (Mimosa); sumac plants (Rhus), especially Sicilian sumac (Rhus coriaria), in particular the fruits; hops (Humlulus); fruit, e.g., apples (Malus), pears (Pryus), strawberries (Fragaria), raspberries (Rubus idaeus), blackberries (Rubus sect. Rubus), cranberries (Vaccinium vitis-idaea), bananas (Musa), grapes (Vitis vinifera), peaches (Prunus persica), quinces (Cydonia oblonga) and/or plums (Prunus domestica); coffee beans (e.g., Coffea arabica or Coffea canephor); tea plants (Camellia sinensis) such as green tea (e.g., Japanese, Chinese and/or Indian teas), in particular the leaves of sencha green tea, black tea, in particular the leaves of Darjeeling, white tea, in particular the leaves of China Oolong white tea, and/or rooibos (Aspalathus linearis); cloves (Syzygium aromaticum), preferably the buds and/or leaves thereof, yarrow (Achillea millefolium); black cohosh (Actaea racemosa); bearberry (Arctostaphylos uva-ursi); tarragon (Artemisia dracunculus), tormentil (Potentilla erecta); and/or peat. Furthermore, for the, in particular comminuted and/or dried, plants or plant constituents, the following may be used: chebulic myrobalan, mango tree (Mangifera indica), in particular the bark, fruit pulp and kernel, Elaeocarpus (Elaecoarpus ganitrus), in particular the outer bark, jute mallow (Corchorus olitorius), red amaranth (Amaranthus cruentus), cowpea (Vigna unguiculata), chard (Beta vulgaris var. cicla.), Greek mountain tea (Sideritis raseri), American witch-hazel (Hamamelis virginiana), in particular the bark, Aleppo oak, dyer's oak (Quercus infectoria), currants (Vitis vinifera apyrena), chicory coffee (Cichorium intybus), carob powder (Ceratonia siliqua), green oat (Avena sativa L.), elm bark (Ulmus), chokeberry pomace (Aronia melanocarpa), linden bark (Tilia), elder bark (Sambucus nigra), lapacho bark (Tabebuia impetiginosa), chicory (Cichorium intybus), amla (Phyllanthus emblica), Chinese blackberry (Rubus suavissimus), pomegranate (Punica granatum L.), sage (Salvia), oregano (Origanum vulgare), cloudberry (Rubus chamaemorus) and date palms (Phoenix). From the yarrow (Achillea millefolium), black cohosh (Actaea racemosa), bearberry (Arctostaphylos uva-ursi), tarragon (Artemisia dracunculus) and tormentil (Potentilla erecta), preferably the whole plant is used for extraction. In the case of betel nut palm (Areca catechu) and the cashew tree (Anacardium occidentale), the seeds are preferably used for extraction.

In a preferred embodiment, the method according to the disclosure according to the first embodiment further comprises (step f)) separating the, in particular comminuted, plants or plant constituents from the product mixture obtained according to step e), in particular following step e), preferably by way of a sieve.

Additionally or alternatively, the method according to the disclosure according to the first embodiment may further comprise (step g)) concentrating, in particular by evaporation, the product mixture containing gallic acid obtained according to step e) or f), in particular following step e) or f) and/or before step h).

Moreover, the method according to the disclosure according to the first embodiment may further additionally or alternatively comprise (step h)) treating the product mixture according to step e) or f) with the at least one polar organic solvent, in particular ethanol and/or methanol, and/or heat-treating the product mixture according to step e) or step f) or g). The treatment with the polar organic solvent, in particular ethanol, or, in particular and, the additional heat treatment consistently allow(s) the enzyme tannase to be deactivated. In particular, this is also achieved by treating the product mixture with the at least one polar organic solvent, in particular ethanol, in step h) at temperatures in the range of 40 to 80° C. or above 45 to 80° C., preferably 40 to 75° C. or 48 to 75° C. and particularly preferably 50 to 70° C. For further optimization of method step h), in this step, additionally or alternatively, in particular additionally, the at least one polar organic solvent, in particular ethanol, may be added in an amount in the range of 15 to 70% by weight, preferably in the range of 20 to 65% by weight, particularly preferably in the range of 25 to 60 and in particular in the range of 40 to 60% by weight, in each case based on the total weight of the polar organic solvent and the water or the aqueous system. Step h), in particular the treatment with a polar solvent, in particular ethanol, consistently allows both the enzyme to be deactivated and phytoproteins and/or polysaccharides to be denatured and precipitated.

In an expedient embodiment, steps f) and g) occur in sequence. Moreover, steps f) and h) may be combined while omitting step g), or steps g) and h) may be combined while omitting step f). In a particularly preferred embodiment, method step e) is followed by method steps f), g) and h), the sequence f)/g)/h) being preferred.

In a preferred embodiment of the method according to the disclosure according to the first embodiment, the treated product mixture according to step h) is filtered, for example using a filter having a mesh size in the range of 0.10 to 1.0 μm, preferably in the range of 0.25 to 1.0 μm. In this way, a clear extract is generally obtained.

In many cases, it has also proved expedient to subsequently remove the polar organic solvent added in step h) from the treated product mixture according to step h) or from the filtered product mixture according to step i) (step j).

The extraction treatment according to step e) is preferably performed at temperatures in the range of 20 to 45° C. or 30 to 43° C., with temperatures in the range of 35 to 40° C., in particular in the range of 36 to 38° C., being particularly preferred.

Further disclosed herein is a method for preparing an extract containing gallic acid (also referred to as a second embodiment of the method according to the disclosure), comprising:
1) providing, in particular comminuted and/or, in particular and, dried, plants or plant constituents, containing tannins, in particular tannic acid,
2) providing the enzyme tannase (IUB 3.1.1.20),
3) providing at least one polar organic solvent, in particular ethanol,
4) optionally comminuting or further comminuting the plants or plant constituents according to step 1),
5) performing an extraction treatment on the, in particular dried and/or comminuted, plants or plant constituents according to step 1) and/or, in particular or, 4) by way of a mixture containing the polar organic solvent, in particular ethanol, and water to obtain a product mixture containing polyphenols,
6) separating the, in particular comminuted, plants or plant constituents from the product mixture according to step 5) to obtain a liquid extract containing the polyphenols, in particular by way of a sieve,
7) removing the polar organic solvent, in particular ethanol, from the liquid extract according to step 6) to obtain an aqueous extract containing the polyphenols,
8) treating the aqueous extract according to step 7) with the enzyme tannase (IUB 3.1.1.20) to obtain an aqueous system containing gallic acid.

Advantageous results are, in particular, obtained by the method according to the second embodiment by performing the extraction treatment according to step 5) at temperatures in the range of 50 to 70° C., in particular in the range of 55 to 65° C.

In a preferred embodiment, the second embodiment variant of the method according to the disclosure is also characterized in that in step 5), ethanol is added in an amount in the range of 60 to 90% by weight, preferably in the range of 70 to 85% by weight, in each case based on the total weight of ethanol and the water.

In the second embodiment of the method according to the disclosure, the treatment of the aqueous extract according to step 8) with the enzyme tannase is preferably carried out at temperatures in the range of 20 to 45° C., preferably 30 to 43° C., particularly preferably 35 to 40° C., in particular in the range of 36 to 38° C.

Surprisingly, it has been shown that a particularly efficient method is also achieved by the, in particular ground, preferably comminuted, plants or plant constituents being immobilized in step e) according to the first embodiment or step 5), respectively, according to the second embodiment, in particular immobilized in a sieve support.

The extraction treatment according to step e) in the first embodiment or step 5) in the second embodiment preferably comprises or is a percolation process.

Particularly high yields or concentrations of gallic acid can also be achieved with the methods according to the disclosure by adjusting the amount of tannase in step e) according to the first embodiment or step 5) according to the second embodiment in the range of 0.1 U/g to 100.0 U/g, preferably in the range of 0.2 U/g to 50.0 U/g, based on the amount of plants or plant constituents.

It has also proved to be particularly practical to concentrate the aqueous extract containing gallic acid obtained according to step i) or j) according to the first embodiment or the aqueous system containing gallic acid according to step 8) according to the second embodiment, in particular by evaporation, to obtain an aqueous concentrate. This aqueous concentrate is preferably a so-called spissum extract. Said aqueous concentrate or said spissum extract has in particular a Brix value in the range of 1° to 100° Brix, preferably in the range of 10° to 70° Brix and particularly preferably in the range of 20° to 50° Brix. Very particularly preferably, concentrates having Brix values in the range of 20° to 40° Brix, in particular in the range of 25° to 35° Brix, are aimed for or obtained in the concentrating step. The determination of degrees Brix is familiar to a person skilled in the art. A liquid has a Brix value of 1° when the liquid has the same density as a solution of 1 g of saccharose in 100 g of saccharose/water solution, and a Brix value of 10° when the density of the liquid is that of a solution of 10 g of saccharose in 100 g of saccharose/water solution. Thus, a figure indicated in degrees Brix means that the density of the measured liquid corresponds to the density of a solution of saccharose in water that contains as many grams of saccharose per 100 g of solution as the figure in degrees.

Additionally disclosed herein is an aqueous concentrate containing gallic acid, in particular obtained according to the first embodiment, the dry-weight amount used for said aqueous concentrate being in the range of 5 to 80% by weight, preferably in the range of 10 to 50% by weight and particularly preferably in the range of 15 to 40% by weight, in each case based on the total weight of the aqueous concentrate. Said aqueous concentrates are particularly suitable for producing foodstuffs and food supplements, in particular those in the form of beverages.

Dry weight within the meaning of the disclosure is understood to be the dry weight obtained by removing all aqueous and non-aqueous solvents from the extracts that can be obtained according to the methods according to the disclosure.

In particular, those aqueous concentrates are preferred which are obtained either according to the first embodiment of the method according to the disclosure or according to the second embodiment of the method according to the disclosure, those aqueous concentrates being particularly preferred which can be obtained according to the method according to the first embodiment.

In particular, the aqueous concentrates according to the disclosure, in particular obtained according to the first embodiment, have a pH in the range of 1.5 to 5.5, preferably in the range of 1.75 to 4.5 and particularly preferably in the range of 2.0 to 4.0, or are adjusted to a pH in the range of 1.5 to 5.5, preferably in the range of 1.75 to 4.5 and particularly preferably in the range of 2.0 to 4.0.

In preferred aqueous concentrates according to the disclosure, in particular obtained according to the first embodiment, the amount of gallic acid is at least 10 g/kg dry weight, preferably at least 50 g/kg dry weight and particularly preferably at least 75 g/kg dry weight. Those aqueous concentrates are particularly expedient in which gallic acid is present in an amount in the range of 10 to 250 g/kg dry weight, preferably in the range of 50 to 200 g/kg dry weight and particularly preferably in the range of 75 to 175 g/kg dry weight, in each case based on the total weight of the aqueous concentrate.

Suitable aqueous concentrates according to the disclosure, in particular obtained according to the first embodiment, preferably have a relative density [20/20] in the range of 1.001 to 1.4000, preferably in the range of 1.010 to 1.2500. The relative density [20/20] is the quotient of the density of the tested sample (measured at 20° C.) and the density of water at 20° C. The measurement is taken in particular by way of a flexural oscillator according to § 35 LMBG 36.0 3a (German Foodstuffs and Commodities Act in the version applicable on the date of filing of the present application) (Determination of the relative density d 20/20 of wort and beer (flexural oscillator method)). Particularly suitable aqueous concentrates are characterized by a total phenol content in the range of 5 to 350 g GAE/kg, preferably in the range of 50 to 300 g GAE/kg and particularly preferably in the range of 75 to 250 g GAE/kg and in particular in the range of 125 to 200 g GAE/kg, in each case based on the dry weight of the extract.

The total phenol content can be determined according to the method according to DIN ISO 14502-1:2007-11 (Determination of substances characteristic of green and black tea—Part 1: Content of total polyphenols in tea—Colorimetric method using Folin-Ciocalteu reagent).

Furthermore, disclosed herein is a foodstuff, for example a beverage, and a food supplement, containing or formed from the aqueous concentrate according to the present disclosure.

The present disclosure is accompanied by the surprising finding that, when the extraction methods according to the disclosure are used according to the first embodiment or according to the second embodiment, but in particular according to the first embodiment, high yields of gallic acid can be obtained efficiently and inexpensively from a wide variety of plant materials or components thereof, meaning that aqueous concentrates having a very high gallic acid concentration can be obtained. It was surprisingly found that the aqueous concentrates according to the disclosure in particular have a Trolox-equivalent antioxidant capacity in the range of 500 to 6,000 μmol/g, preferably in the range of 1,000 to 5,000 μmol/g and particularly preferably 2,000 to 4,000 μmol/g, in each case based on the dry weight of the extract. The Trolox-equivalent antioxidant capacity can be expediently determined by the DPPH (2,2-diphenyl-1-picrylhydrazyl) assay. This determination method is familiar to a person skilled in the art and is described, for example, by O. Sharma and T. N. Bhat in “DPPH antioxidant assay revisited,” Food Chemistry 113(4):1202-1205, April 2009, and by A. Karioti et al. in “Composition and antioxidant activity of the essential oils of Xylopia aethiopica (Dun) A. Rich. (Annonaceae) leaves, stem bark, root bark, and fresh and dried fruits, growing in Ghana,” J. Agric. Food Chem. 29;52(26):8094-8, December 2004. For example, in this method, 10 mg of the stable radical DPPH can be dissolved in 100 ml methanol, mixed with the particular sample extract or the standard (Trolox), and then incubated for 30 min at room temperature, and the absorbance is subsequently determined photometrically at 517 nm. The antioxidant potential of the samples can then be determined from a linear relationship between absorbance and the standard concentrations. The results can be calculated as Trolox equivalents or as micromoles of Trolox/g of the sample and in relation to the dry weight. With the described method, an absorbance difference is determined, from which the antioxidant capacity of the tested substance can be determined by comparison with the reduction in absorbance caused by Trolox at different concentrations.

The aqueous concentrates according to the disclosure can be directly used for producing foodstuffs, in particular beverages, and food supplements.

The features of the disclosure disclosed in the above description and in the claims may be essential either individually or in any desired combination for carrying out the various embodiments of the disclosure.

The various embodiments described above can be combined to provide further embodiments. All of the foreign patents, foreign patent applications, and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for preparing an extract containing gallic acid, comprising the steps of:
a) providing plants or plant constituents containing tannins,
b) providing the enzyme tannase,
c) providing at least one polar organic solvent,
d) optionally comminuting the plants or plant constituents according to step a), and
e) performing an extraction treatment on the plants or plant constituents obtained according to step a) and/or step d) by way of water or an aqueous system in the presence of the enzyme tannase to obtain a product mixture containing gallic acid.

2. The method according to claim 1, wherein the extraction treatment according to step e) is carried out at temperatures in a range of 20° C. to 45° C.

3. The method according to claim 1, wherein the plants or plant constituents are immobilized in step e) in a sieve support.

4. The method according to claim 1, wherein the extraction treatment according to step e) comprises a percolation process.

5. The method according to claim 1, wherein the extraction treatment according to step e) is carried out at temperatures in a range of 35° C. to 40° C.

6. The method according to claim 1, wherein the amount of tannase in step e) is in a range of 0.1 U/g to 100.0 U/g, based on the amount of plants or plant constituents.

7. The method according to claim 1, further comprising:
f) separating the plants or plant constituents from the product mixture obtained according to step e), and/or
g) concentrating the product mixture obtained according to step e) or f), containing gallic acid, and/or
h) treating the product mixture according to step e) or f) with the at least one polar organic solvent and/or heat-treating the product mixture according to step e) or f) or g), and
i) optionally filtering the treated product mixture according to step h), and/or
j) optionally removing the polar organic solvent added in step h) from the treated product mixture according to step h) or the filtered product mixture according to step i).

8. The method according to claim 7, wherein the treatment of the product mixture with the at least one polar organic solvent, according to step h) is carried out at temperatures in the range of 40° C. to 75° C.

9. The method according to claim 7, wherein in step h), the at least one polar organic solvent is added in an amount in the range of 15 to 70% by weight based on the total weight of the polar organic solvent and the water or the aqueous system.

10. The method according to claim 7, further comprising concentrating the aqueous system containing gallic acid according to step i) or step j) to obtain an aqueous concentrate.

11. A method for preparing an extract containing gallic acid, comprising the steps of:
1) providing dried plants or plant constituents containing tannins,
2) providing the enzyme tannase,
3) providing at least one polar organic solvent,
4) optionally comminuting the plants or plant constituents according to step 1),
5) performing an extraction treatment on the plants or plant constituents according to step 1) and/or step 4) with a mixture containing the polar organic solvent and water to obtain a product mixture containing polyphenols,
6) separating the plants or plant constituents from the product mixture according to step 5) to obtain a liquid extract containing the polyphenols,
7) removing the polar organic solvent from the liquid extract according to step 6) to obtain an aqueous extract containing the polyphenols, and
8) treating the aqueous extract according to step 7) with the enzyme tannase to obtain an aqueous system containing gallic acid.

12. The method according to claim 11, wherein the plants or plant constituents are immobilized in step 5) in a sieve support.

13. The method according to claim 11, wherein the extraction treatment according to step 5) comprises a percolation process.

14. The method according to claim 11, wherein the amount of tannase in step 8) is in a range of 0.1 U/g to 100.0 U/g, based on the amount of plants or plant constituents.

15. The method according to claim 11, further comprising:
concentrating the aqueous system containing gallic acid according to step 8) to obtain an aqueous concentrate.

16. The method according to claim 15, wherein the aqueous concentrate has a Brix value in a range of 1° to 100° Brix.

17. The method according to claim 11, wherein the extraction treatment according to step 5) is carried out at temperatures in a range of 50° C. to 70° C.

18. The method according to claim 11, wherein the treatment of the aqueous extract according to step 8) with the enzyme tannase is carried out at temperatures in a range of 35° C. to 40° C.

19. The method according to claim 11, wherein in step 5), ethanol is added in an amount in the range of 60 to 90% by weight based on the total weight of ethanol and the water.

20. An aqueous concentrate containing gallic acid obtained or obtainable according to the method of claim 1 comprising:
a dry weight in the range of 5 to 80% by weight based on the total weight of the aqueous concentrate.

21. The aqueous concentrate according to claim 20, comprising:
a pH in a range of 1.5 to 5.5.

22. The aqueous concentrate according to claim 20, comprising:
an amount of gallic acid of at least 10 g/kg dry weight and/or an amount of gallic acid in a range of 10 to 250 g/kg dry weight based on the total weight of the aqueous concentrate.

23. An aqueous concentrate according to claim 20, comprising:
a relative density [20/20] in a range of 1,001 to 1,400 determined by way of a flexural oscillator method according to § 35 LMBG 36.0 3a (Determination of the relative density d 20/20 of wort and beer).

24. A foodstuff containing or made from an aqueous concentrate according to claim 15.

25. A foodstuff containing or made from an aqueous concentrate containing gallic acid obtained or obtainable according to the method of claim 6, the aqueous concentrate comprising a dry weight in the range of 5 to 80% by weight based on the total weight of the aqueous concentrate.
